(19) United States
(12) Patent Application Publication
(10) Pub. No.: US 2015/0126968 A1
(43) Pub. Date: May 7, 2015

(54) FLUID DELIVERY DEVICES AND METHODS

(71) Applicant: PALO ALTO RESEARCH CENTER INCORPORATED, Palo Alto, CA (US)

(72) Inventors: Ramkumar Abhishek, Mountain View, CA (US); Felicia Linn, San Jose, CA (US)

(73) Assignee: PALO ALTO RESEARCH CENTER INCORPORATED, Palo Alto, CA (US)

(21) Appl. No.: 14/071,929

(22) Filed: Nov. 5, 2013

(51) Int. Cl. A61M 31/00

(52) U.S. Cl. CPC A61M 31/002
USPC 604/514; 604/244; 604/131; 604/500

(57) ABSTRACT

Devices and methods are provided for fluid delivery. The device may include a housing having an outer surface which includes a porous membrane, a fluid reservoir disposed in the interior region of the main housing and formed at least in part by a wall structure, a puncture mechanism operable to puncture the wall structure and form a fluidic path between the fluid reservoir and one or more channels in fluid communication with the porous membrane, and a positive displacement mechanism operable, following puncture of the wall structure, to drive a fluid out of the fluid reservoir, through the one or more channels, and into the porous membrane.

### FIELD

The present disclosure is generally in the field of fluid delivery devices and methods, and more particularly to devices and methods for the controlled delivery of one or more fluids, including but not limited to implantable drug delivery devices.

### BACKGROUND

Controlled fluid delivery is desirable in a variety of applications, including drug delivery. However, it would be desirable to provide improved devices and methods to independently store and effectively deliver one or more fluids from a single device to a target area. For example, it would be desirable to provide a delivery system capable of separately storing two or more fluids onboard a device, yet including shared means for controlled fluid release from the device in a compact, space-efficient device.

### SUMMARY

In one aspect, a fluid delivery device is provided, including: (i) a main housing having an interior region and having an outer surface which includes a porous membrane, (ii) a first fluid reservoir disposed in the interior region of the main housing, the first fluid reservoir being formed at least in part by a first wall structure and containing a first fluid, (iii) a first puncture mechanism operable to puncture the first wall structure and form a fluidic path between the first fluid reservoir and one or more first channels, which are in fluid communication with the porous membrane, and (iv) a first positive displacement mechanism operable, following puncture of the first wall structure, to drive the first fluid out of the first fluid reservoir, through the one or more first channels, and into the porous membrane. The porous membrane may be configured to distribute the first fluid to an area adjacent to and outside of the outer surface of the main housing.

In another aspect, an implantable drug delivery device for controlled release of two or more drugs to an intralumenal tissue surface is provided, the device including: (i) a main housing configured for intralumenal deployment in a patient, the main housing having an outer surface which includes a porous membrane, (ii) a first fluid reservoir within the main housing and containing a first fluid which includes a first drug, (iii) a second fluid reservoir within the main housing and containing a second fluid which includes a second drug, (iv) a first puncture mechanism operable to puncture the first fluid reservoir and faun a fluidic path between the first fluid reservoir and one or more first channels, which are in fluid communication with the porous membrane, (v) a second puncture mechanism operable to puncture the second fluid reservoir and form a fluidic path between the second fluid reservoir and one or more second channels, which are in fluid communication with the porous membrane, and (vi) at least one positive displacement mechanism operable to (a) drive the first fluid out of the first fluid reservoir, through the one or more first channels, and into the porous membrane following puncture of the first fluid reservoir, and (b) drive the second fluid out of the second fluid reservoir, through the one or more second channels, and into the porous membrane following puncture of the second fluid reservoir. The porous membrane may be operable to distribute the first and second drugs to an intralumenal tissue surface adjacent to the outer surface of the main housing.

In yet another aspect, a method of controlled delivery of a fluid to a target area is provided, the method including: (i) positioning a fluid delivery device adjacent to the target area, wherein the fluid delivery device includes: (a) a first fluid reservoir defined by a first fluid reservoir housing, the first fluid reservoir containing a first fluid, (b) a first wall structure forming at least a portion of the first fluid reservoir housing, and (c) a porous membrane forming an outer surface of the fluid delivery device, (ii) puncturing the first wall structure with a first puncture mechanism to provide a fluidic path between the first fluid reservoir and one or more first channels in fluid communication with the porous membrane, and (iii) driving the first fluid out of the first fluid reservoir and into the one or more first channels, such that the first fluid is delivered to the target area from the device via the porous membrane.

### BRIEF DESCRIPTION OF THE DRAWINGS

FIG. 1A is a cross-sectional view of one embodiment of a fluid delivery device having a single fluid reservoir, in a tissue lumen, prior to puncturing the wall structure.

FIG. 1B is a cross-sectional view of the fluid delivery device of FIG. 1A, after puncturing the wall structure and prior to driving the fluid out of the fluid reservoir.

FIG. 1C is a cross-sectional view of the fluid delivery device of FIG. 1A, while driving the fluid out of the fluid reservoir and into the channels to deliver the fluid to the tissue surface via the porous membrane.

FIG. 2A is a cross-sectional view of one embodiment of a fluid delivery device having two fluid reservoirs, prior to puncturing the wall structure of either reservoir.

FIG. 2B is a cross-sectional view of the fluid delivery device of FIG. 2A, after puncturing the wall structure of the first fluid reservoir and while driving the fluid out of that reservoir.

FIG. 2C is a cross-sectional view of the fluid delivery device of FIG. 2A, after puncturing the wall structure of the second fluid reservoir and while driving the fluid out of that reservoir.

FIG. 3A is an exploded perspective view of one embodiment of a fluid delivery device having two fluid reservoirs and two independent sets of channels in fluid communication with a porous membrane.

FIG. 3B is a perspective view of the fluid delivery device of FIG. 3A.

FIG. 4A is a perspective view of one embodiment of an implantable drug delivery device having a porous membrane sidewall.

FIG. 4B is an exploded perspective view of the drug delivery device of FIG. 4A.

FIG. 5A is a cross-sectional view of one embodiment of a fluid delivery device having a single fluid reservoir, in a tissue lumen, prior to puncturing the wall structure.

FIG. 5B is a cross-sectional view of the fluid delivery device of FIG. 5A, after puncturing the wall structure and prior to driving the fluid out of the fluid reservoir.

FIG. 5C is a cross-sectional view of the fluid delivery device of FIG. 5A, while driving the fluid out of the fluid reservoir and into the channels to deliver the fluid to the tissue surface via the porous membrane.

### DETAILED DESCRIPTION

The fluid delivery devices and methods described herein provide for the storage and controlled delivery of one or more fluids from a single device. The devices are advantageously configured to separately store and isolate the fluid(s), thereby minimizing the risk of contamination, and to precisely dispense the fluid(s) according to a desired release timing profile. For example, these devices and methods may be used to deliver drugs to a patient in vivo. The devices and methods disclosed herein can significantly increase the accuracy and efficiency of delivering multiple fluids, in a space-efficient manner, e.g., with minimal duplication of the fluid delivering components, and without unwanted mixing of the fluids to be released.

In certain embodiments, the fluid delivery devices include a porous membrane to distribute the fluid(s) to the area adjacent the device. However, the devices disclosed herein may also be constructed without the porous membrane. For example, a device may have a common gate or valve other than a porous membrane, which is configured to distribute the fluid(s) to the area adjacent the device.

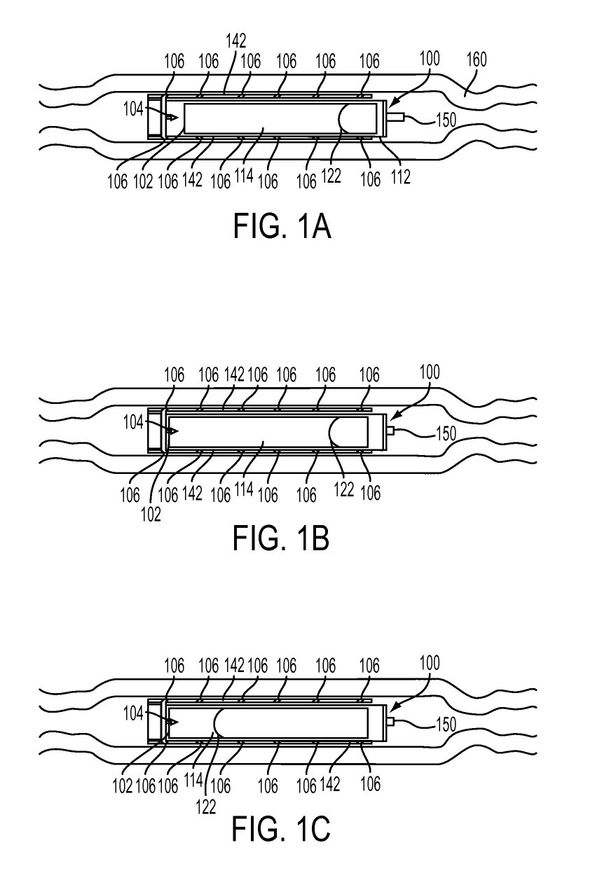

In one aspect, a fluid delivery device is provided. As shown in FIG. 1A, in one embodiment the device 100 includes a main housing 112 having an interior region and having an outer surface which includes a porous membrane 142. A first fluid reservoir 114 is disposed in the interior region of the main housing 112, and is formed at least in part by a first wall structure 102. The first fluid reservoir 114 contains a first fluid. The device 100 also includes a first puncture mechanism 104 operable to puncture the first wall structure 102 and form a fluidic path between the first fluid reservoir 114 and one or more first channels 106, which are in fluid communication with the porous membrane 142. The device 100 also includes a first positive displacement mechanism 122 operable, following puncture of the first wall structure 102, to drive the first fluid out of the first fluid reservoir 114, through the one or more first channels 106, and into the porous membrane 142. The porous membrane 142 is configured to distribute the first fluid to an area adjacent to and outside of the outer surface of the main housing 112.

In another aspect, a method of controlled fluid delivery to a target area is provided. In certain embodiments, as shown in FIGS. 1A-1C the method includes: (i) positioning a fluid delivery device 100 adjacent to the target area, (ii) puncturing the first wall structure 102 with a first puncture mechanism 104 to provide a fluidic path between the first fluid reservoir 114 and one or more first channels 106 in fluid communication with the porous membrane 142, and (iii) driving the first fluid out of the first fluid reservoir 114 and into the one or more first channels 106, such that the first fluid is delivered to the target area from the device 100 via the porous membrane 142.

Various embodiments and features of the fluid delivery devices and methods are described in greater detail hereinafter.

### Main Housing

The device includes a main housing having an interior region and an outer surface. In certain embodiments, the outer surface of the main housing includes a porous membrane. Generally, these devices are configured to deliver one or more fluids to an area adjacent to and outside of the outer surface of the main housing.

The main housing of the device may be shaped and dimensioned for a specific fluid delivery application. For example, as shown in FIG. 1A, the device 100 may be an implantable medical device shaped and dimensioned for intralumenal deployment into a human or animal subject. The term “intralumenal,” as used herein, refers to placement within a body cavity, channel, tube, or the like, having a mucosal wall. The term includes, but is not limited to, sites in the reproductive tract, such as intravaginal, cervical, or intrauterine, and the gastrointestinal tract, such as intrarectal.

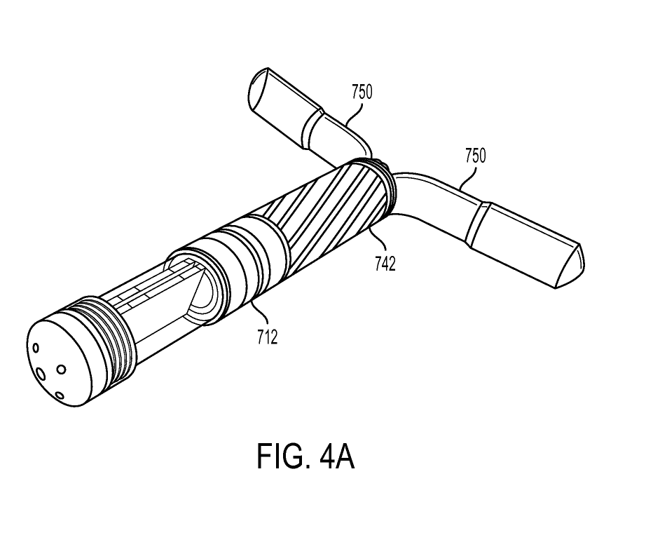

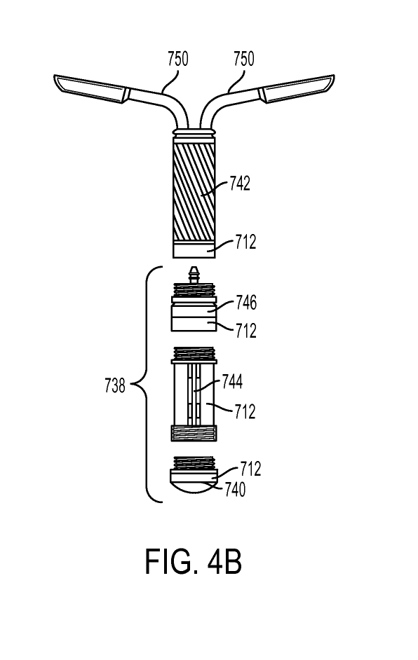

The housing configuration may be based upon the particular lumenal site and human or animal anatomical considerations, for deployment with minimal discomfort to the patient. In certain embodiments, the device may be placed within the lumen by insertion into the lumen via an exterior body orifice. Accordingly, in certain embodiments, the housing is shaped and dimensioned to allow insertion and placement, i.e., deployment, of the device within the intended lumen via the exterior body orifice. For example, the housing may be shaped and dimensioned for vaginal, cervical, uterine, or rectal insertion and placement. As shown in FIGS. 4A and 4B, the housing 712 may include an elongated, substantially cylindrical outer surface and have wing-like portions, or arms, 750 extending therefrom. For example, this configuration may be appropriate for vaginal device deployment in livestock, such as cattle, sheep, etc.

The materials of construction, size, shape, surface features, and other characteristics of the housing may be configured such that the device can be deployed into the lumen, retained securely in the lumen during operation of the device for an extended period of time, and retrieved from the lumen following operation of the device or when otherwise desired to be removed. For example, the device may be removed between the delivery of individual drug formulations, following the delivery of several drug formulations, or following the completion of a course of treatment of multiple drug formulations. The device may be deployed until the drug formulation payload is depleted.

In certain embodiments, the housing is formed of a biocompatible material. Moreover, the housing material may be resistant to degradation in the mucosal environment of the lumen. Examples of suitable housing materials include stainless steel, titanium, and biocompatible polymers, such as polypropylene, polyethylene, or other common polymers, such as nylon having a biocompatible outer layer, e.g., silicone. The housing material may include a coating to enhance biocompatibility and/or operation of the device.

### Reservoirs and Contents

At least one reservoir is disposed within the main housing of the device and contains a fluid for delivery from the device. The reservoir(s) may be shaped and dimensioned to fit within the main housing and may be formed of materials that are suitable for storing the fluid to be contained therein. Examples of suitable reservoir materials include stainless steel, titanium, and biocompatible polymers, such as polypropylene, polyethylene, or other common polymers, such as nylon having a biocompatible outer layer, e.g., silicone. In certain embodiments, the reservoir may be formed at least in part by the inner surface of the main housing.

As shown in FIG. 1A, the reservoir 114 is formed at least in part by a first wall structure 102. The first wall structure may include a puncturable material. Examples of suitable wall structure materials include elastomeric polymers or other self-sealing materials, such as silicone, PTFE, and synthetic and natural rubber, and combinations thereof.

In certain embodiments, the device includes two or more reservoirs, with each reservoir containing a fluid to be delivered and being formed at least in part by a wall structure. In particular embodiments, the fluids are drugs that are selected to work in concert, and beneficially are delivered in parallel or series, for example in a separated or overlapping schedule. In certain embodiments, multiple reservoirs may contain a similar fluid to be delivered in multiple administrations. For example, the device may include multiple reservoirs containing the same drug to be administered in discrete doses to a patient.

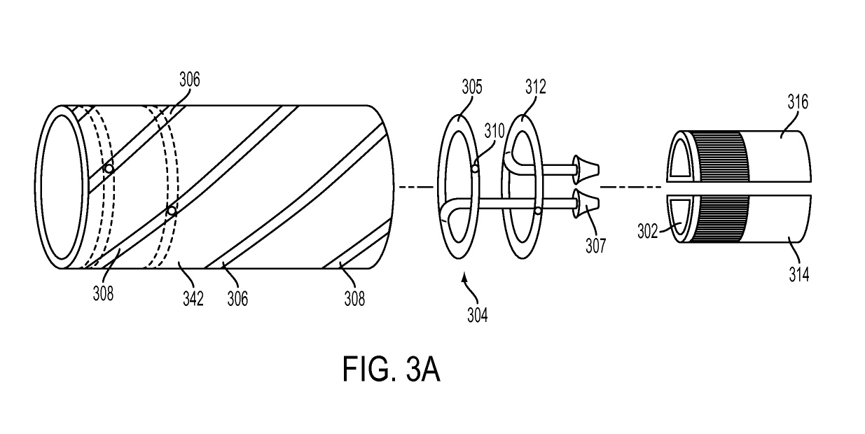

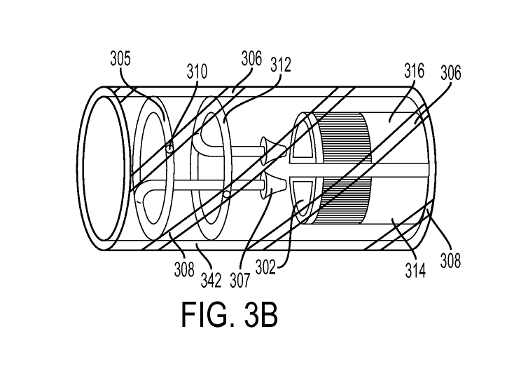

The reservoirs may be disposed within the housing such that they are parallel to the housing and/or each other. The reservoirs may each be defined by an inner surface of an elongated annular tube. The reservoirs may also have a combined shape similar to that of the housing and be configured such that the reservoirs occupy a majority of the volume of the housing. In certain embodiments, the reservoirs are elongated and have a circular cross-sectional shape. In certain embodiments, the reservoirs together have a circular cross-sectional shape, with each reservoir having a cross-sectional shape that forms a portion of the circular shape. In one embodiment, as shown in FIGS. 3A and 3B, each of two reservoirs 314, 316 has a semi-circular cross-sectional shape such that the reservoirs together form a circular cross-sectional shape. Other cross-sectional shapes are also envisioned. In certain multi-reservoir embodiments, at least two of the reservoirs are configured to have different volumes.

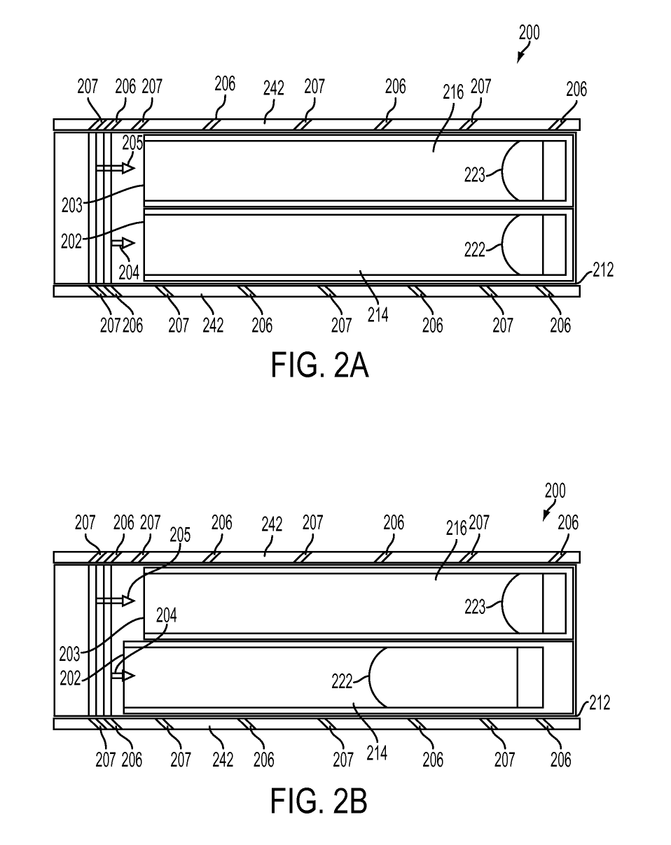

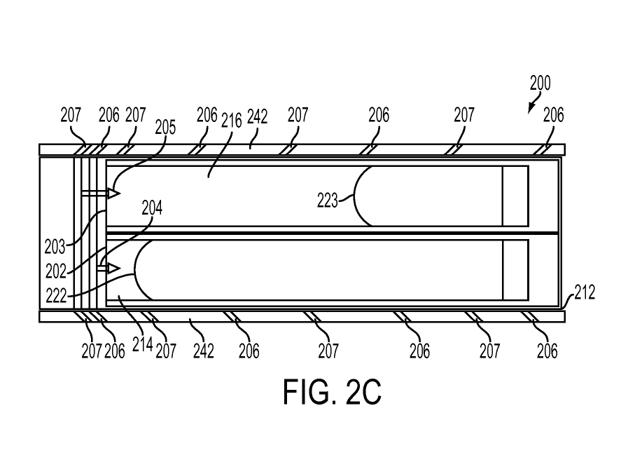

As shown in FIG. 2A, the device 200 includes a first reservoir 214 containing a first fluid and a second reservoir 216 containing a second fluid. Both reservoirs 214, 216 are disposed in the interior region of the main housing 212. The first reservoir 214 is formed in part by a first wall structure 202 and the second reservoir 216 is formed in part by a second wall structure 203. In certain embodiments, the first and second fluid reservoirs each include an elongated annular tube with a closed end wall.

In certain embodiments, each reservoir has an actuation end and an opposed release end. The actuation ends may be operably connected to an actuation system. The release ends may include the wall structure that is configured to be punctured by the puncture mechanism. The actuation end of each reservoir includes a positive displacement mechanism that is operable, following puncture of the reservoir's wall structure, to drive the fluid out of the reservoir. As shown in FIGS. 1A-1C, the device 100 includes a first positive displacement mechanism 122 operable, following puncture of the first wall structure 102, to drive the first fluid out of the first fluid reservoir 114, through the one or more first channels 106, and into the porous membrane 142. In embodiments having multiple reservoirs, each reservoir may be associated with a separate positive displacement mechanism, as shown in FIGS. 2A-2C, or two or more reservoirs may share a positive displacement mechanism.

In certain embodiments, the positive displacement mechanism includes an expandable membrane. For example, the expandable membrane may include an inflatable balloon structure that expands or inflates to drive the fluid out of the reservoir. The balloon structure may be an elastomer, such as latex, nitrile or urethane based, or it may be a collapsed balloon, such as a thin metallized polymer sheet, e.g., polyester or polyethylene.

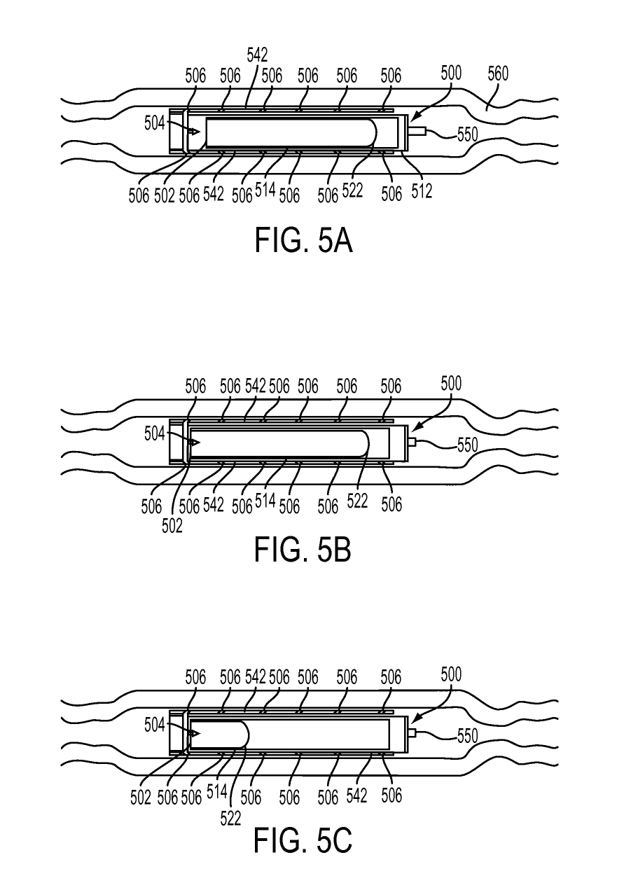

In other embodiments, as shown in FIGS. 5A-5C, the fluid is contained within a balloon 522 disposed in the reservoir 514, and the positive displacement mechanism includes a gas generating mechanism configured to generate a gas to deflate the balloon and displace the first fluid. As shown in FIG. 5A, in one embodiment the device 500 includes a main housing 512 having an interior region and having an outer surface which includes a porous membrane 542. A first fluid reservoir 514 is disposed in the interior region of the main housing 512, and is formed at least in part by a first wall structure 502. The first fluid reservoir 514 at least partially contains balloon 522, which contains a first fluid. The device 500 also includes a first puncture mechanism 504 operable to puncture the first wall structure 502 and form a fluidic path between the first fluid reservoir 514 and/or the balloon 522 and one or more first channels 506, which are in fluid communication with the porous membrane 542. The device 500 also includes positive displacement mechanism operable, following puncture of the first wall structure 502 (shown in FIG. 5B), to generate a gas in communication with the balloon 522, to deflate the balloon 522 and drive the first fluid out of the first fluid reservoir 514, through the one or more first channels 506, and into the porous membrane 542, as shown in FIG. 5C. The porous membrane 542 is configured to distribute the first fluid to an area adjacent to and outside of the outer surface of the main housing 512.

Alternatively, the positive displacement mechanism may include a plug or plunger structure configured to advance through the reservoir and drive the fluid out of the reservoir. For example, the plugs may sealingly engage with, and slide with respect to, the inner walls of the reservoirs. The plugs may function as pistons.

For example, the devices described herein may include one or more of the reservoir and/or main housing features described in U.S. patent application Ser. Nos. 13/629,124 and 13/629,159, the disclosures of which are incorporated herein by reference in pertinent part.

### Release Structure

In embodiments, the device is configured to deliver the fluid(s) to a target area adjacent the outer surface of the main housing. To initiate the release of the fluid from a reservoir, the wall structure of the reservoir is punctured by the puncture mechanism and the positive displacement mechanism is actuated to drive the fluid out of the reservoir. The puncture mechanism is operable to puncture the wall structure and form a fluidic path between the reservoir and one or more channels. In certain embodiments, the channels are in fluid communication with a porous membrane configured to distribute the fluid to an area adjacent to and outside of the outer surface of the main housing. For example, the one or more channels may extend along the length of the porous membrane, such as in a helical or linear shape.

In certain embodiments, the puncture mechanism includes a channel in fluid communication with a needle tip oriented to puncture the wall structure of the reservoir. As shown in FIGS. 1A-1C, the first puncture mechanism 104 includes a channel in fluid communication with a needle tip oriented to puncture the first wall structure 102. In certain embodiments, as shown in FIGS. 3A and 3B, the first puncture mechanism 304 includes an annular channel 305 in fluid communication with a needle tip 307 oriented to puncture the first wall structure 302.

As shown in FIGS. 2A-2C, the device 200 includes first and second puncture mechanisms 204, 205 operable to puncture the first and second wall structures 202, 203, respectively, and form a fluidic path between the first and second fluid reservoirs 214, 216, and the first and second sets of channels 206, 207, respectively, which are in fluid communication with the porous membrane 242. First and second positive displacement mechanisms 222, 223 are operable, following puncture of the first and second wall structures 202, 203, respectively, to drive the first and second fluids out of the first and second fluid reservoirs 214, 216, through the first and second sets of channels 206, 207, and into the porous membrane 242. The porous membrane 242 is configured to distribute both the first and second fluids to the area adjacent to and outside of the outer surface of the main housing 212. In one embodiment, as shown in FIGS. 3A and 3B, the one or more first channels 306 are distinct from the one or more second channels 308, such that the fluidic paths for the first and second fluids may be isolated from one another. For example, separate sets of channels may decrease contamination.

In certain embodiments, the device includes a mechanism configured to allow a user to selectively cause the first puncture mechanism to puncture the first wall structure. As shown in FIGS. 1A-1C, the device 100 may include a push button 150 disposed on the outer surface of main housing 112, such that it is accessible by a user. For example, the device may include a first tension loaded mechanism configured to advance the first reservoir towards the puncture mechanism. In certain embodiments, the device includes a single push button configured to cause all of the reservoirs in the device to be punctured, in series or parallel. For example, the device may be configured to puncture the reservoirs in series, according to a predetermined timing schedule.

In certain embodiments, the puncture mechanism includes a channel in fluid connection with the needle tip and configured to form a fluidic path between the channels disposed in or adjacent to the porous membrane. As shown in FIGS. 3A and 3B, the channel 305 of the first puncture mechanism 304 may be an annular channel. In certain embodiments, the channel of the puncture mechanism is sized and shaped to be adjacent the inner surface of the main housing. As shown in FIGS. 3A and 3B, the channel 305 of the first puncture mechanism 304 includes a radial aperture 310 in fluid communication with the one or more first channels 306, which are in fluid communication with the porous membrane 342. In certain embodiments, each puncture mechanism includes one or more apertures or fluid connections that provide the fluid communication with apertures or fluid connections in the channels of the porous membrane.

As shown in FIGS. 3A and 3B, the first and second annular channels 305, 312 may be positioned together in a stacked and/or offset arrangement within the interior region of the main housing.

For example, the needle tip of the puncture mechanism may be constructed of drug-compatible, biocompatible plastics or metals, such as stainless steel, and combinations thereof. The needle tip may be stiff or soft. For example, the needle tip may be stiff to assist in the puncturing process, or may be soft to assist in plugging the mechanism to prevent leakage once engaged with the reservoir. The channel of the puncture mechanism, as well as the channels in communication with the porous membrane, may be constructed of any drug-compatible, biocompatible tubing material, such as silicone, PTFE, polypropylene, and polyethylene, and/or metals, such as stainless steel.

Once the wall structure of the reservoir is breached by the puncture mechanism, i.e., by advancing the reservoir toward the puncture mechanism or advancing the puncture mechanism toward the reservoir, the positive displacement mechanism is actuated to drive the fluid out of the reservoir, through the fluidic path of the puncture mechanism and into the associated channels, which are in fluid communication with the porous membrane. For example, the lumenal surface of each of the one or more channels adjacent to or within the porous membrane may be porous, or include a plurality of outlets for providing a fluidic path between the channels and the porous membrane.

The porous membrane, or other common gate or valve structures used in place of or in addition to the porous membrane, may function to redirect or spread the fluid across a greater area of the region adjacent the device. For example, in a drug delivery device, the porous membrane sidewall may be configured to diffuse and distribute the drug formulations released from the reservoirs to the lumenal tissue. The porous membrane sidewall may be configured to distribute the drug formulations driven from the reservoirs to a tissue area adjacent the porous membrane sidewall when the device is deployed intralumenally in a human or animal subject.

The porous membrane may advantageously maximize the area which is exposed to the dispensed fluids by diffusing the fluids and distributing them across a large porous area on the outer surface of the device. The surface area of the porous membrane, and therefore the adjacent surface area to receive the fluid, may be adjusted depending upon the targeted fluid and delivery site. The porous membrane may also provide a reproducible wettable surface condition that reduces variability in the administered fluid and variability in the delivered fluid doses. Such features may be beneficial over conventional technologies having individual ports for each fluid to be dispensed. Such conventional devices may suffer from random delivery patterns and a lack of control over fluid and site exposure, resulting in highly variable dosing.

In one embodiment, the porous membrane operates as a fluidic valve. For example, the porous membrane may have a pore structure and chemistry such that a positive pressure is required to initiate flow of the fluid(s) through the porous membrane. This thresholding pressure may be tuned by controlling the average pore size of the membrane's pore structure, as well as the contact angle of the fluid on the surface of the membrane material. For example, the porous membrane sidewall may be a fluidic valve configured such that a critical threshold pressure from about 0.1 psi to about 100 psi is required to distribute the fluids to the area adjacent the outer surface of the main housing.

The pore structure may be any microstructure representative of an open pore structure. This may be a single layer of pores that expend from one surface of the membrane through to the opposing surface of the membrane. Alternatively, the pore structure may be a randomly packed structure of interconnected pores or a highly ordered, closed packed pores structure. For example, the porous membrane may have an average pore size from about 0.2 μm to about 25 μm.

In certain embodiments, the porous membrane acts as an aseptic barrier. For example, the porous membrane may be configured to substantially prohibit infiltration into the device of bacteria having a size in excess of the effective average pore size of the porous membrane.

The porous membrane may be constructed of a polycarbonate, polypropylene, PFTE, or polyethylene membrane, or any combination of laminates thereof. The membrane may also be constructed of two or more dissimilar materials serving different functions outlined above. For example, a PTFE and polyethylene laminate structure may be used to achieve effective fluid solution spreading, antimicrobial delivery, and valving. Alternatively, a composite material may be constructed to achieve these desired functions for fluids having differing wetting characteristics. This may be achieved, for example, by using interwoven porous sheets constructed of a predetermined ratio of hydrophobic to hydrophilic materials.

In certain embodiments, upon generation of a positive pressure via the positive displacement mechanism, the fluids are driven from the reservoirs and through the porous membrane sidewall. Once the pressure is reduced, the wetting condition will become thermodynamically unfavorable and flow will stop.

In certain embodiments, the porous membrane provides a surface that is in primary contact with body tissue and therefore is composed of biocompatible materials. For example, the porous membrane may include a polypropylene membrane. Other suitable porous membrane materials include, but are not limited to, polyethersulfone, polycarbonate, polyethylene terephthalate, polyvinylidene fluoride, mixed cellulose ester, nylon 6,6, polytetrafluoroethylene, and combinations thereof.

In certain embodiments, the porous membrane substantially surrounds the main housing about the one or more reservoirs. For example, the porous membrane may be cylindrical. In certain embodiments, the porous membrane includes a first portion adjacent the release ends (i.e., the ends near the puncture mechanisms) of the reservoirs and a second portion adjacent the actuation ends of the reservoirs such that the drug formulations are distributed from both the first and second portions of the porous membrane. For example, the one or more channels may extend from a first end (i.e., at the release end) of the porous membrane to the opposed second end (i.e., at the actuation end) of the porous membrane.

For example, the devices described herein may include one or more of the release structure and/or multiple fluid delivery features described in U.S. patent application Ser. Nos. 13/629,124 and 13/629,159, the disclosures of which are incorporated herein by reference in pertinent part.

### Actuation System

In certain embodiments, the device includes one or more actuation systems which are configured to actuate the positive displacement mechanism(s) and in turn drive the fluid(s) from the reservoir(s). The one or more actuation systems may also be configured to actuate puncture of the wall structures of the reservoirs, i.e., by advancing the reservoir toward the puncture mechanism or advancing the puncture mechanism toward the reservoir. In one embodiment, a single actuation system may be configured to actuate multiple positive displacement mechanisms, so as to drive the first fluid from the first reservoir and drive the second fluid from the second reservoir, in series or parallel. In another embodiment, multiple actuation systems may be configured to actuate multiple positive displacement mechanisms so as to drive multiple fluids from multiple reservoirs.

The one or more actuation systems may be operably connected to the actuation ends of each of the reservoirs. Generally, each actuation system is configured to drive the fluid in the reservoir via a positive displacement process. The term “positive displacement,” as used herein, refers to any process whereby the drug formulations are dispensed from the drug delivery device under force provided within each reservoir. Accordingly, the term does not refer to the passive, chemical diffusion of the drug formulations out of the reservoir, although passive diffusion may contribute to release of the drug formulations from the porous membrane.

As shown in FIG. 4B, the actuation system 738 may include a power source 740, control circuitry 744, and an actuation mechanism 746. Embodiments having more than one actuation system may include multiple actuation mechanisms and a shared power source and control circuitry. Alternatively, embodiments having more than one actuation system may include multiple individual actuations systems, each having a power source, control circuitry, and actuation mechanism.

The power source may be any source of mechanical, electrical power or electromechanical power. The power source may include one or more batteries or fuel cells. The control circuitry may be configured to control the actuation system of the device, and thereby control the timing of release of the fluids. For example, the control circuitry may selectively transmit electrical and/or mechanical power to the actuation mechanism, positively displacing the fluid in the reservoirs. The control circuitry may be configured to control the timing of delivery of the fluids by applying the necessary electrical signals to the actuation mechanism. The control circuitry may be programmable or it may be pre-programmed to deliver the fluids in accordance with a prescribed (predetermined) release schedule.

The actuation mechanism may include fluid-volume displacement, mechanical displacement, osmotic swelling displacement, electrostatically-induced compression, piezoelectric actuation, thermally/magnetically induced phase transformation, or combinations thereof, to drive the fluids from the reservoirs via positive displacement. For example, the actuation mechanism may include one or more of the actuation mechanisms described in U.S. patent application Ser. Nos. 13/629,124 and 13/629,159, the disclosures of which are incorporated herein by reference in pertinent part.

In certain embodiments, the one or more actuation systems are each configured to generate a displacement fluid in operable communication with an inflatable balloon structure within each reservoir. For example, the actuation system may include an electrolytic cell having a cathode and an anode which is in contact with water or an aqueous solution to generate a gas, such as oxygen, thus providing a source of positive displacement to drive the fluids out of the reservoirs.

In certain embodiments, the actuation system further includes a wireless receiver for receiving wireless control signals from a separate, detached transmitting device. For example, the fluid delivery device may be deployed into the lumen of a patient by the patient, physician, veterinarian, or the like, and thereafter, the patient, physician, veterinarian, or the like, may actuate the release of the fluids using the transmitting device to transmit control signals to the deployed device. Furthermore, in some embodiments, the receiver and transmitting device may both be transceivers capable of transmitting and receiving control signals and other communications from each other. For example, the receiver and transmitting device may be configured to allow for wireless programming and re-programming of the drug dosing regimen, including the dosage, timing of drug release, etc. Accordingly, in certain embodiments, the transceiver may transmit data relevant to the operation of the device, such as data regarding the fluids already administered, the release schedule, the amount of fluids remaining in the reservoirs, and the remaining battery charge, as well as data relevant to the environment of the deployment site, such as data detected or measured by an integral sensor. In some embodiments, the actuation system may also be wirelessly powered.

In certain embodiments, the device may is configured for wireless operation, e.g., following deployment in a human or animal subject. In such cases, the device includes appropriate telemetry components as known in the art. For example, actuation of the fluid dispensing may be done from a remote controller, e.g., external to a human or animal subject. Generally, the telemetry (i.e. the transmitting and receiving) is accomplished using a first coil to inductively couple electromagnetic energy to a matching/corresponding second coil. The means of doing this are well established, with various modulation schemes such as amplitude or frequency modulation used to transmit the data on a carrier frequency. The choice of the carrier frequency and modulation scheme will depend on the location of the device and the bandwidth required, among other factors. Other data telemetry systems known in the art also may be used. In another case, the device is configured to be remotely powered, or charged. For example, the device may include a transducer for receiving energy wirelessly transmitted to the device, circuitry for directing or converting the received power into a form that can be used or stored, and if stored, a storage device, such as a rechargeable battery or capacitor. In still another case, the device is both wirelessly powered and wirelessly controlled.

### Drug Formulations

In certain embodiments, the fluid delivery device is an implantable drug delivery device for the controlled release of one or more drugs to an intralumenal tissue surface of a patient. In such embodiments, one or more drug formulations are contained within the device reservoirs for delivery to the mucosal tissue. In one embodiment, two or more drug formulations are disposed within two reservoirs for release to a subject.

Various drug formulations may be administered from the drug delivery device. The drug formulations within each reservoir may each include the same drug, may each include different drugs, or may be some combination of more than one similar drug and more than one different drug. For example, the first drug formulation may include a different drug than the second drug formulation. For example, the first and third drug formulations may both include the same drug, and second drug formulations may include a different drug than the first and third drug formulations. In certain embodiments, the device may be used to deliver a battery of drug formulations for a combination therapy, prophylaxis, or for another specific treatment, such as may be useful in animal husbandry.

In one embodiment, the device is used to deliver a fixed time artificial insemination treatment to a human or animal subject. In certain embodiments, the first drug formulation includes a gonadotropin-releasing hormone, the second drug formulation includes a prostaglandin, and the third drug formulation includes a gonadotropin-releasing hormone. In one embodiment, the device also includes a fourth drug formulation which includes a progestin. Variations of the drugs and sequences are envisioned.

In embodiments, the drug formulations include one or more proteins or peptides. For example, in some embodiments, the drug delivery device may be used to administer hormones or steroids. including, but not limited to, follicle stimulating hormone, parathyroid hormone, luteinizing hormone, gonadotropin-releasing hormone (GnRH), estradiol, progesterone, melatonin, serotonin, thyroxine, triiodothyronine, epinephrine, norepinephrine, dopamine, antimullerian hormone, adiponectin, adrenocorticotropic hormone, angiotensinogen, angiotensin, antidiuretic hormone, atrial-natriuretic peptide, calcitonin, cholecystokinin, corticotropin-releasing hormone, erythropoietin, gastrin, ghrelin, glucagon, growth hormone-releasing hormone, human chorionic gonadotropin, human placental lactogen, growth hormone, inhibin, insulin, insulin-like growth factor, leptin, melanocyte stimulating hormone, orexin, oxytocin, prolactin, relaxin, secretin, somatostatin, thrombopoietin, thyroid-stimulating hormone, thyrotropin-releasing hormone, cortisol, aldosterone, testosterone, dehydroepiandrosterone, androstenedione, dihydrotestosterone, estrone, estriol, calcitriol, calcidiol, prostaglandins, leukotrienes, prostacyclin, thromboxane, prolactin releasing hormone, lipotropin, brain natriuretic peptide, neuropeptide Y, histamine, endothelin, enkephalin, renin, and pancreatic polypeptide.

In some embodiments, the drug delivery device may be used to administer cytokine signaling molecules or immunomodulating agents that are used in cellular communication. These molecules commonly comprise proteins, peptides, or glycoproteins. Cytokine signaling molecules include, for example, the four a-helix bundle family which include the IL-2 subfamily (e.g., erythropoietin (EPO) and thrombopoietin (THPO)), the interferon (IFN) subfamily and the IL-10 subfamily. Cytokine signaling molecules also include the IL-1, IL-18, and IL-17 families.

In some embodiments, the drug delivery device may be used to administer drug formulations for pain management, including, but not limited to, corticosteroids, opioids, antidepressants, anticonvulsants (antiseizure medications), non-steroidal anti-inflammatory drugs, COX2 inhibitors (e.g., rofecoxib and celecoxib), ticyclic antidepressants (e.g., amitriptyline), carbamazepine, gabapentin and pregabalin, codeine, oxycodone, hydrocodone, diamorphine, and pethidine.

In some embodiments, the drug delivery device may be used to administer cardiovascular drug formulations. Examples include B-type natriuretic peptide (BNP), atrial natriuretic peptide (ANP), atrial natriuretic factor (ANF), atrial natriuretic hormone (ANH), and atriopeptin. Cardiovascular drug formulations that may be administered by the device also include, for example, antiarrhythmic agents, such as Type I (sodium channel blockers), including quinidine, lidocaine, phenytoin, propafenone; Type H (beta blockers), including metoprolol; Type III (potassium channel blockers), including amiodarone, dofetilide, sotalol; Type IV (slow calcium channel blockers), including diltiazem, verapamil; Type V (cardiac glycosides), including adenosine and digoxin. Other cardiacvascular drug formulations that may be administered by the device include ACE inhibitors, such as, for example, captopril, enalapril, perindopril, ramipril; angiotensin II receptor antagonists, such as, for example, candesartan, eprosartan, irbesartan, losartan, telmisartan, valsartan; beta blocker; and calcium channel blocker.

The drug formulations may be formulated with one or more pharmaceutically acceptable excipients as needed to facilitate the drug's storage in and release from the device. In one embodiment, the drug may be in a liquid solution or suspension. The drug may be in the form of microparticles or nanoparticles. The solvent or carrier may be aqueous or organic. For example, the devices and methods described herein may further include a reconstitution mechanism as described in U.S. patent application Ser. No. 13/629,184, the disclosure of which is incorporated herein by reference in pertinent part.

In some embodiments, the drug formulations may include components that are degradable by the enzymes present in the fluid secreted by the mucosal tissue. For example, certain amino acids present in drug formulations may be degraded by the enzymes present in fluid secreted by the mucosal tissue. Accordingly, the devices and methods described herein may further include one or more of the permeation enhancement mechanisms described in U.S. Patent Application Publications No. 2011/0087195, No. 2011/0087192, and No. 2011/0087155, the disclosures of which are incorporated herein by reference in pertinent part.

### Methods

Methods are provided for controlled delivery of fluid(s) to a target area. The devices may include any combination of the device feature described herein. In certain embodiments, as shown in FIGS. 1A-1C the method includes: (i) positioning a fluid delivery device 100 adjacent to the target area, (ii) puncturing the first wall structure 102 with a first puncture mechanism 104 to provide a fluidic path between the first fluid reservoir 114 and one or more first channels 106 in fluid communication with the porous membrane 142, and (iii) driving the first fluid out of the first fluid reservoir 114 and into the one or more first channels 106, such that the first fluid is delivered to the target area from the device 100 via the porous membrane 142.

In certain embodiments, the methods include deploying a drug delivery device into the mucosal lumen of a human or animal patient. For example, the subject may be a mammalian animal (e.g., cow, sheep, horse, pig, or dog). The methods include various medical and veterinary therapies, as well as animal husbandry applications. The lumen may be, for example, a vagina, cervix, uterus, bladder, or rectum. The device may be adapted to contact essentially any mucosal tissue surface. The device may be placed in the lumen by inserting the device through an exterior orifice of the patient into the lumen. In some embodiments, the device may be in a form that may be orally administered for delivery of a drug via the mucosal tissue of the gastrointestinal tract.

In certain embodiments, the first wall structure is punctured prior to positioning the fluid delivery device adjacent to the target area. For example, as shown in FIGS. 1A-1C, a user may press the button 150, to actuate puncturing of the wall structure 102, prior to positioning the device within a bodily lumen 160. In other embodiments, the puncturing of one or more wall structures may be actuated wirelessly after the device has been positioned.

In certain embodiments, driving the fluid out of the fluid reservoir includes generating a gas to inflate the balloon within the fluid reservoir and displace the fluid. In other embodiments, the device further includes a balloon containing the first fluid disposed in the first fluid reservoir, and driving the first fluid out of the first fluid reservoir includes generating a gas to deflate the balloon and displace the first fluid. For example, in devices having multiple reservoirs, the fluid in the first reservoir may be completely or partially released from the first reservoir before the release of the fluid from the second reservoir.

As illustrated in FIG. 1, the fluid delivery device 100 may be placed in a lumen 160. The device may be held in place by frictional engagement between the mucosal tissue and the main housing. As shown in FIGS. 4A-4B, arms 750 may be provided to facilitate retention of the device within the mucosal lumen. The fluids may then be driven out of the reservoirs and into the porous membrane 742 from which the drug formulations are then released to the surrounding area. The actuation of the actuation system may be controlled by the control circuitry 744. The device may thereafter be removed from the lumen. For example, the devices described herein may include one or more of the retention features described in U.S. patent application Ser. No. 13/742,203, the disclosure of which is incorporated herein by reference in pertinent part.

The fluid delivery devices may include any of the device features described herein. For example, the device may include a microcontroller configured to control the actuation system, and thereby control the timing of the release of the fluids.

### Applications/Uses

The fluid delivery devices and methods may be used for any applications in which controlled fluid delivery to a target area is needed. For example, the fluid delivery devices and methods may be used for various medical and therapeutic applications in human and animal subjects, as well as in animal husbandry. In certain embodiments, the fluids are drug formulations.

In one embodiment, an implantable drug delivery device for the controlled release of two or more drugs to an intralumenal tissue surface is provided. The device includes a main housing configured for intralumenal deployment in a patient and having an outer surface which includes a porous membrane. First and second fluid reservoirs are contained within the main housing, and contain a first drug and a second drug, respectively. The first and second drugs may be the same drug formulation or different drug formulations. A first puncture mechanism is operable to puncture the first fluid reservoir and form a fluidic path between the first fluid reservoir and one or more first channels, which are in fluid communication with the porous membrane. A second puncture mechanism is operable to puncture the second fluid reservoir and form a fluidic path between the second fluid reservoir and one or more second channels, which are also in fluid communication with the porous membrane. At least one positive displacement mechanism is operable to (i) drive the first fluid out of the first fluid reservoir, through the one or more first channels, and into the porous membrane following puncture of the first fluid reservoir, and (ii) drive the second fluid out of the second fluid reservoir, through the one or more second channels, and into the porous membrane following puncture of the second fluid reservoir. The porous membrane is operable to distribute the first and second drugs to an intralumenal tissue surface adjacent to the outer surface of the main housing.

In some embodiments, the drug delivery device may be used to treat infertility or provide a fixed time artificial insemination (FTAI) treatment in a female subject. For example, the drug delivery device may be placed in the vagina (or uterus, or other part of the birth canal) of a female subject. The drug delivery device may then deliver follicle stimulating hormone to induce ovulation in the female subject. In some embodiments, the drug delivery device may be configured to deliver a plurality of hormones, including follicle stimulating hormone, luteinizing hormone, gonadotropin-releasing hormone separately, or in combination, in appropriate sequences, at appropriate times, and in pharmacologically appropriate amounts. The device may also dispense estradiol to regulate natural hormone production in the female subject. The appropriate dosing schedule and amounts may be determined by one in the field of reproductive pharmacology.

Compared to traditional FTAI treatments, the methods described herein may require only device implantation and removal at the time of artificial insemination, and result in a reduction in time spent driving, herding and chuting cattle. The methods also result in improved ovulation quality and quantity due to the reduction in handling, stress, and systemic cortisol levels of the subjects. The methods also reduce the number of medical supplies needed, as a single device delivery the series of FTAI drugs.

In another embodiment, the drug delivery device may be used to treat insulin dependent diabetes (Type I diabetes) in a subject. The drug delivery device may be placed within a lumen of the subject. The drug delivery device may then deliver insulin (Humulin R, Novolin R), insulin isophane (Humulin N, Novolin N), insulin lispro (Humalog), insulin aspart (NovoLog), insulin glargine (Lantus) or insulin detemir (Levemir) to the patient at a selected time or times.

In another embodiment, the drug delivery device may be used to treat diabetes mellitus (Type II diabetes) in a subject. The drug delivery device may be placed within a lumen of the subject. The drug delivery device may then deliver exenatide to the patient at a selected time or times.

In another embodiment, the drug delivery device may be used to treat breast or ovarian cancer in a subject. The drug delivery device may be placed within a lumen of the subject, such as the vagina for a female subject. The drug delivery device may then deliver abraxane (or other drug effective in the treatment or management of cancer) to the patient at a selected time or times.

In another embodiment, the drug delivery device may be used to treat HIV/AIDS in a subject. The drug delivery device may be placed within a lumen of the subject. The drug delivery device may then deliver Abacavir (ABC) or Cidofovir (or other drug effective in the treatment or management of HIV/AIDS) to the patient at a selected time or times. The device also may be used to treat other sexually transmitted diseases.

In another embodiment, the drug delivery device may be used to treat genital herpes in a subject. The drug delivery device may be placed within a lumen of the subject, such as within the vagina of a female subject. The drug delivery device may then deliver acyclovir, famciclovir, or valacyclovir (or other drug effective in the treatment or management of genital herpes) to the patient at a selected time or times.

In another embodiment, the drug delivery device may be used to treat diabetes insipidus in a subject. The drug delivery device may be placed within a lumen of the subject. The drug delivery device may then deliver desmopressin (or other drug effective in the treatment or management of diabetes insipidus) to the patient at a selected time or times.

In another embodiment, the drug delivery device may be used to treat osteoporosis in a subject. The drug delivery device may be placed within a lumen of the subject, such as within the vagina of a female subject. The drug delivery device may then deliver ibandronate, calcitonin, or parathyroid hormone (or other drug effective in the treatment or management of osteoporosis) to the patient at a selected time or times.

Overall, the devices and methods described herein provide a means to deliver multiple fluids through a common distribution means (e.g., gate, valve, porous membrane). The devices take advantage of the common porous membrane, while providing the ability to engage the reservoir-membrane fluidic connection just before use. Thus, this engaging mechanism allows the reservoirs to remain unbreached during shipping and on the shelf, thus maintaining the chemical and biological integrity of the fluids contained therein, such as drugs. Moreover, the device structure allows each fluid to be delivered via individual and isolated channels, thereby enabling independent access to the common membrane for each fluid dispensed and reducing the potential for contamination.

It will be appreciated that various of the above-disclosed and other features and functions, or alternatives thereof, may be desirably combined into many other different devices, methods, or applications. Various presently unforeseen or unanticipated alternatives, modifications, variations, or improvements therein may be subsequently made by those skilled in the art which are also intended to be encompassed by the following claims.

We claim:

1. A fluid delivery device, comprising:
a main housing having an interior region and having an outer surface which comprises a porous membrane;
a first fluid reservoir disposed in the interior region of the main housing, the first fluid reservoir being formed at least in part by a first wall structure and containing a first fluid;
a first puncture mechanism operable to puncture the first wall structure and form a fluidic path between the first fluid reservoir and one or more first channels, which are in fluid communication with the porous membrane; and
a first positive displacement mechanism operable, following puncture of the first wall structure, to drive the first fluid out of the first fluid reservoir, through the one or more first channels, and into the porous membrane,
wherein the porous membrane is configured to distribute the first fluid to an area adjacent to and outside of the outer surface of the main housing.

2. The device of claim 1, wherein the first positive displacement mechanism comprises an inflatable balloon.

3. The device of claim 1, wherein:
the first fluid is contained within a balloon disposed in the first fluid reservoir, and
the first positive displacement mechanism comprises a gas generating mechanism configured to generate a gas to deflate the balloon and displace the first fluid.

4. The device of claim 1, wherein the first puncture mechanism comprises an annular channel in fluid communication with a needle tip oriented to puncture the first wall structure.

5. The device of claim 1, further comprising a tension loaded mechanism configured to allow a user to selectively cause the first puncture mechanism to puncture the first wall structure.

6. The device of claim 1, wherein the outer surface and porous membrane are cylindrical.

7. The device of claim 6, wherein the one or more first channels extend from a first end of the porous membrane to an opposed second end of the porous membrane.

8. The device of claim 1, further comprising:
a second fluid reservoir disposed in the interior region of the main housing, the second fluid reservoir being formed at least in part by a second wall structure and containing a second fluid;
a second puncture mechanism operable to puncture the second wall structure and form a fluidic path between the second fluid reservoir and one or more second channels, which are in fluid communication with the porous membrane; and
a second positive displacement mechanism operable, following puncture of the second wall structure, to drive the second fluid out of the second fluid reservoir, through the one or more second channels, and into the porous membrane,
wherein the porous membrane is configured to distribute the second fluid to an area adjacent to and outside of the outer surface of the main housing.

9. The device of claim 8, wherein the one or more second channels are distinct from the one or more first channels.

10. The device of claim 8, wherein:
the first and second fluid reservoirs each comprise an elongated annular tube with a closed end wall, and
the first and second wall structures respectively comprise the closed end walls of the first and second fluid reservoirs.

11. The device of claim 10, wherein:
the first puncture mechanism comprises a first annular channel in fluid communication with a first needle tip configured to puncture the first wall structure,
the second puncture mechanism comprises a second annular channel in fluid communication with a second needle tip configured to puncture the second wall structure, and
the first and second annular channels are positioned together in a stacked and/or offset arrangement within the interior region.

12. The device of claim 1, wherein a lumenal surface of each of the one or more first channels adjacent to or within the porous membrane is porous.

13. The device of claim 1, wherein the porous membrane comprises a fluidic valve configured such that a critical threshold pressure from about 0.1 psi to about 100 psi is required to distribute the first fluid to the area adjacent to and outside of the outer surface of the main housing.

14. The device of claim 1, wherein the porous membrane comprises an aseptic barrier configured to substantially prohibit infiltration into the device of bacteria having a size in excess of an average pore size of the porous membrane.

15. The device of claim 1, wherein the porous membrane comprises a membrane material selected from the group consisting of polypropylene, polyethylene, polytetrafluoroethylene, and combinations thereof.

16. The device of claim 1, wherein the porous membrane has an average pore size from about 0.2 μm to about 25 μm.

17. The device of claim 1, wherein the first fluid comprises a drug.

18. The device of claim 17, which is an implantable medical device shaped and dimensioned for intravaginal deployment.

19. An implantable drug delivery device for controlled release of two or more drugs to an intralumenal tissue surface, the device comprising:
a main housing configured for intralumenal deployment in a patient, the main housing having an outer surface which comprises a porous membrane;
a first fluid reservoir within the main housing and containing a first fluid which comprises a first drug;
a second fluid reservoir within the main housing and containing a second fluid which comprises a second drug;
a first puncture mechanism operable to puncture the first fluid reservoir and form a fluidic path between the first fluid reservoir and one or more first channels, which are in fluid communication with the porous membrane;
a second puncture mechanism operable to puncture the second fluid reservoir and form a fluidic path between the second fluid reservoir and one or more second channels, which are in fluid communication with the porous membrane; and
at least one positive displacement mechanism operable to (i) drive the first fluid out of the first fluid reservoir, through the one or more first channels, and into the porous membrane following puncture of the first fluid reservoir, and (ii) drive the second fluid out of the second fluid reservoir, through the one or more second channels, and into the porous membrane following puncture of the second fluid reservoir,
wherein the porous membrane is operable to distribute the first and second drugs to an intralumenal tissue surface adjacent to the outer surface of the main housing.

20. A method of controlled delivery of a fluid to a target area, comprising:
positioning a fluid delivery device adjacent to the target area, wherein the fluid delivery device comprises:
a first fluid reservoir defined by a first fluid reservoir housing, the first fluid reservoir containing a first fluid;
a first wall structure forming at least a portion of the first fluid reservoir housing; and
a porous membrane forming an outer surface of the fluid delivery device;
puncturing the first wall structure with a first puncture mechanism to provide a fluidic path between the first fluid reservoir and one or more first channels in fluid communication with the porous membrane; and
driving the first fluid out of the first fluid reservoir and into the one or more first channels, such that the first fluid is delivered to the target area from the device via the porous membrane.

21. The method of claim 20, wherein the first wall structure is punctured prior to positioning the fluid delivery device adjacent to the target area.

22. The method of claim 20, wherein driving the first fluid out of the first fluid reservoir comprises generating a gas to inflate a balloon within the first fluid reservoir and displace the first fluid.

23. The method of claim 20, wherein:
the device further comprises a balloon containing the first fluid, the balloon being disposed in the first fluid reservoir, and
driving the first fluid out of the first fluid reservoir comprises generating a gas to deflate the balloon and displace the first fluid.

24. The method of claim 20, wherein the first fluid comprises a drug and the target area is a mucosal tissue surface of a patient.
